(19) United States
(12) Patent Application Publication
(10) Pub. No.: US 2022/0183659 A1
(43) Pub. Date: Jun. 16, 2022

(54) Earphone Driver And Applications

(71) Applicant: Sonicedge Ltd., Shoham (IL)

(72) Inventor: Mordehai Margalit, Zichron Yaaqov (IL)

(21) Appl. No.: 17/543,972

(22) Filed: Dec. 7, 2021

Related U.S. Application Data
(60) Provisional application No. 63/125,414, filed on Dec. 15, 2020.

(51) Int. Cl. A61B 8/00; H04R 1/10; B06B 3/00; A61B 8/12

(52) U.S. Cl. CPC A61B 8/4494; H04R 1/1016; A61B 8/4281; A61B 8/12; A61B 8/467; B06B 3/00; A61B 8/4455

(57) ABSTRACT

An audio system includes a modulated ultrasound audio driver having at least one ultrasound emitter configured to emit an ultrasound signal; at least one acoustic mixer configured to generate an audio signal from the ultrasound signal; and at least one ultrasound receiver configured to detect an ultrasound signal and generate a corresponding electrical signal. A processing unit is configured to process the electrical signal and estimate one or more physical or biometric parameters from the electrical signal. A method for physical or biometric parameter estimation is also disclosed.

This application claims the priority benefit of U.S. provisional application No. 63/125,414, filed on Dec. 15, 2020, the content of which is hereby incorporated by reference herein.

### BACKGROUND

U.S. Pat. No. 8,861,752 describes a speaker device for generating audio signals from modulated ultrasound which has several unique features; the frequency response is constant across the audio frequencies; very small form factor; low cost; high electrical to acoustical conversion efficiency leading to reduced power consumption by the speaker device. U.S. Pat. No. 10,284,961 describes implementation of the speaker device using micro-electro-mechanical system (MEMS) technology. A speaker device includes a plurality of ultrasound membranes, and corresponding acoustic modulators. The ultrasound used in the speaker device is typically above 100 KHz and hence has significant attenuation in air. However, in earphone or headset applications the speaker device is located within a few cm from the ear including the ear canal and tympanic membrane.

Examples in the prior art describe methods for using ultrasound as a sensing modality related to the ear. As an example, US20100069752 describes a method using ultrasound for detecting ear disorders relating to the viscoelasticity of fluid in the ear. An alternative example US20190261094 describes a method for using ultrasound to detect the proximity of the ear to an earphone. Previous systems have required a dedicated ultrasound source and detector.

In this invention we utilize modulated ultrasound speaker as an ultrasound probe in addition to its audio functionality.

### GLOSSARY

“audio signals” as used in the current disclosure means sound pressure waves ranging from 10 Hz to 45,000 Hz.

“audio generating device”—as used in the current disclosure means a device to generate audio signals.

“acoustic signal” as used in the current disclosure means a device to generate audio or ultrasound (ranging above 20 KHz up to 10 MHz) signals.

“acoustic transducer” as used in the current disclosure means a device to generate audio or ultrasound signals.

“controller” or “electronics integrated circuit”—as used in the current disclosure means a device that receives and outputs analog or digital electrical signals and includes logic or microprocessor units to process the input or output signals

“drive signal”—as used in the current disclosure means an electric analog signal. One or more of the drive signals are used to operate an audio generating device

“analog signal”—as used in the current disclosure means a time varying electric analog signal which can have any voltage or current value within a range of values

“digital signal”—as used in the current disclosure means a time varying electric digital signal which can have either of two voltage or current values.

“audio system” as used in the current disclosure means a system for generating audio signals and in some examples includes one or more audio generating devices and one or more controllers

“background sound signals” or “background noise” as used in the current disclosure means audio signals which are present when the audio system is not operating.

“communication bus” as used in the current disclosure means a means of communicating between one or more devices. Communication buses are any of but not limited to; wire; multiple wires; wireless; optical and others.

“power bus” as used in the current disclosure means a method or providing electrical power to one more device.

### DESCRIPTION OF FIGURES

FIG. 1 is an example of a state of art audio generating device as described in U.S. Pat. No. 8,861,752;

FIG. 2 schematically illustrates anatomy of a typical human ear;

FIG. 3. is an example of an earphone comprising a modulated ultrasound speaker and located near the ear canal;

FIG. 4 is an example of a top view of an audio generating device configured to include an ultrasound source and detector;

FIG. 5 is an example of an US transceiver configured to detect an ultrasound back scatter signal;

FIG. 6 is an example of a method for measuring a physical parameter using ultrasound back reflection;

FIG. 7 is an example of an earphone system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other examples may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure. This disclosure is drawn, inter alia, to methods, apparatus, computer programs, and systems of generating measuring physical or biological related phenomena using ultrasound.

In some examples, the ultrasound is generated and measured by a speaker device that includes a membrane and a shutter. The membrane is configured to oscillate along a first directional path and at a combination of frequencies with at least one frequency effective to generate an ultrasonic acoustic signal. A shutter and blind are positioned proximate to the membrane. In one non limiting example the membrane, the blind, and the shutter may be positioned in a substantially parallel orientation with respect to each other. In other examples the membrane, the blind, and the shutter may be positioned in the same plane and the acoustic signal is transmitted along acoustic channels leading from the membrane to the shutter. In a further example the modulator and or shutter are composed of more than one section.

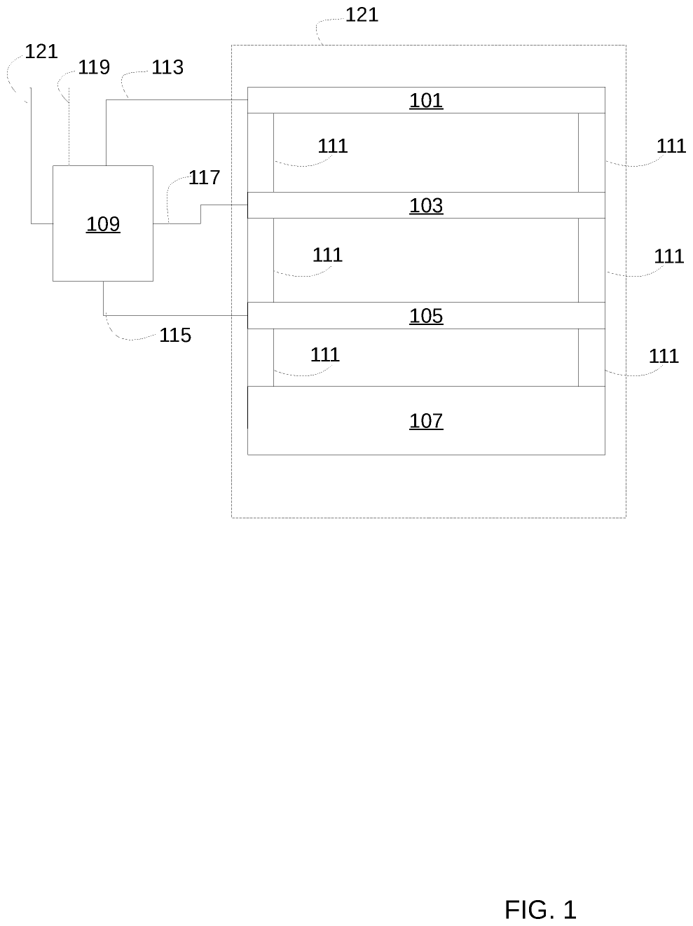

FIG. 1 is an example of a side view of a state of art architecture for a MEMs speaker cell (121). The speaker cell is composed of at least three layers. Membrane (105), which generates an acoustic signal by movement in the direction of arrows (190). Blind (103) and shutter (101) move relative to each other and modulate the acoustic signal. In one example, driving device (109) provides one voltage signal to membrane (15) and a second voltage signal to shutter (101) and the voltage to blind (103) is set at zero or ground. The first and second voltage signals provide the driving force to generate the acoustic sound and the modulation function, respectively. In a further example a speaker device is composed of multiple speaker cells (121).

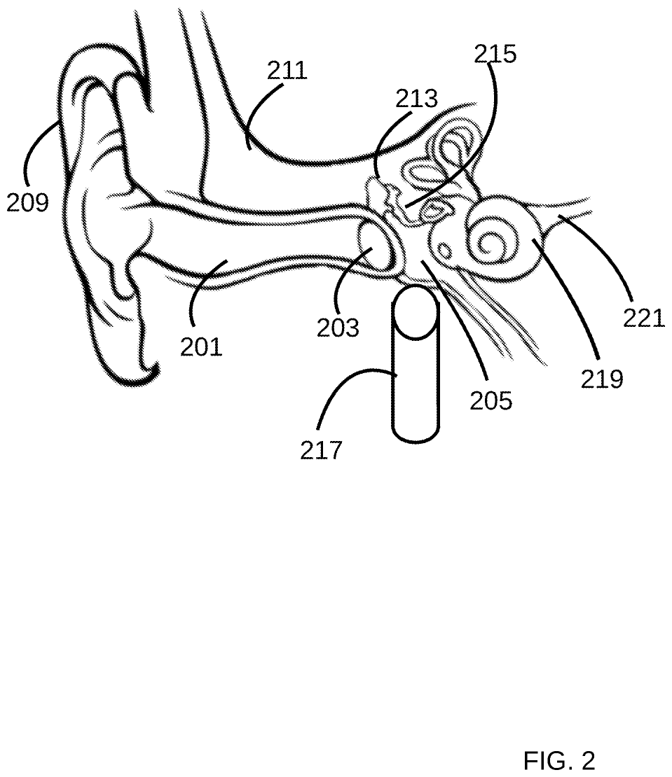

FIG. 2 schematically illustrates anatomy of a typical human ear and includes the pinna (209), ear canal (201), eardrum or tympanic membrane (203), tympanic cavity (205), hammer (213), anvil (215), temporal bone (211), internal jugular vein (217), cochlear (219) and cochlear nerve (221). The ear canal (201) is filled with air and terminated by the tympanic membrane (103). Sound is conducted in air from the pinna (209) to the tympanic membrane (203) where the tympanic membrane (103) moves in response to the local sound pressure. Movement is transferred via the hammer (213) and anvil (215) to the liquid filled cochlear (219). Earphones are designated as receiver in canal for devices located within the ear canal (201), ear buds, in ear monitors, true wireless or speakers, or similar names for devices located in or near the entrance to the ear canal (201) and headphones or headsets for devices located on, or around the pinna (209).

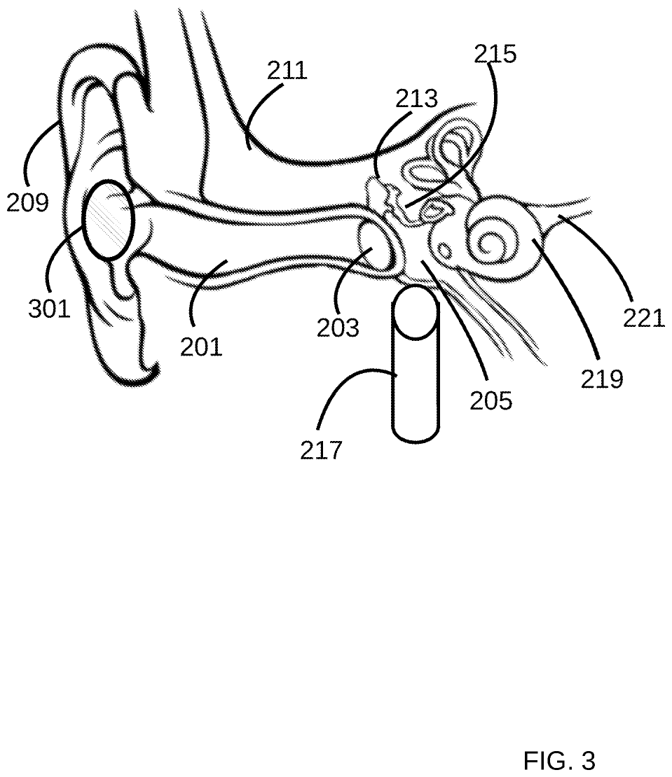

FIG. 3. Is an example of an earphone comprising a modulated ultrasound speaker and located near the ear canal. In one example the modulated ultrasound speaker generates ultrasound at frequencies in the range of frequencies between 50 KHz to 500 KHz. For ultrasound frequencies between 50 KHz to 500 KHz the attenuation in air is 0.01 dB/cm to 0.2 dB/cm. The wavelength of the acoustic wave in this frequency range is between 6.8 mm to 0.6 mm, with an attainable resolution of 1/10 the wavelength. Hence the attenuation of the ultrasound is low and the signal back reflection from the ear canal and tympanic membrane is large enough to detect, measure and extract from the ultrasound back reflections various physical characteristics and their physiological manifestation. Examples of physical or physiological parameters include but are not limited to; distance of speaker from tympanic membrane; volume of ear canal, length or ear canal; heart beat from reflection of veins in ear, or jugular vein, or from vibrations induced by blood flow and manifested in tympanic membrane; effusivity of ear; viscosity of ear fluid; infections or other problems in inner ear; vibrations in tympanic membrane resulting from external noise or internal noise; blood flow; temperature in ear canal and relation to body temperature; proximity to ear, ear canal or tympanic membrane. In one example an ultrasound measurement is conducted in parallel to audio signal generation. In another example, the ultrasound measurement is conducted without an audio signal. In another example

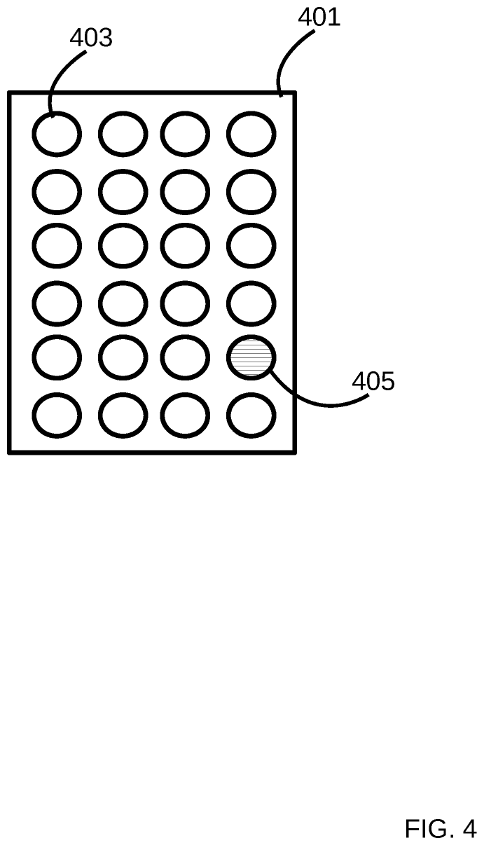

FIG. 4 is an example of a top view of an audio generating device configured to include an ultrasound source and detector. In one example, the audio generating device (401) is manufactured using micro-electromechanical system technology (MEMS) as described in FIG. 1. The audio generating device includes one or more speaker units (403), where each speaker unit is composed of two or more membranes as outlined in FIG. 1. In one example the membranes are configured to operate at frequencies up to 5 MHz. In a further non limiting examples, the resonance frequency of one or more of the membranes is configured to be in the range between 200 to 500 KHz, with a Q factor between 5 to 100. In one example all membranes in an audio generating device are identical. In an alternative example one or more of the membranes (e.g. 405) is configured as either an ultrasound source, ultrasound receiver, or both and designated as US transceiver (405). In one example US transceivers (405) differ from the membranes (403) in resonant frequency, Q factor, horizontal or vertical structure. In a further example US transceiver is electrically connected independently of membrane (403). In one example, an ultrasound signal is generated by one or more membranes (403). The signal is either generated as part of the audio conversion activity or specifically to generate ultrasound backscatter for parameter extraction. In one example, at least one membrane is operated at its resonant frequency with a duty cycle between 40 to 60% to achieve a maximal mechanical movement and generating an ultrasound signal. At least one US transceiver (405) is configured to measure the back scatter of the ultrasound signal generated by at least one membrane (403). Examples of measurements include but are not limited to, time delay from start of signal transmit to start of signal receive, phase delay of received signal compared to transmitted signal, doppler frequency shift of received signal, attenuation of received signal compared to transmitted signal, multiple echoes or backscattering tail of received signal. A controller configured to measure any of or combination of these measurements, can further process the results to extract the physical or physiological data. In a further example the controller is configured with a machine learning or artificial intelligence algorithm and the measurements either processed, or as raw data, are used in a feedback loop, where examples of feedback loops include but are not limited to active noise cancellation, audio manipulation which is dependent on the distance from the tympanic membrane or on the ear canal cavity volume or ear canal cavity leakage.

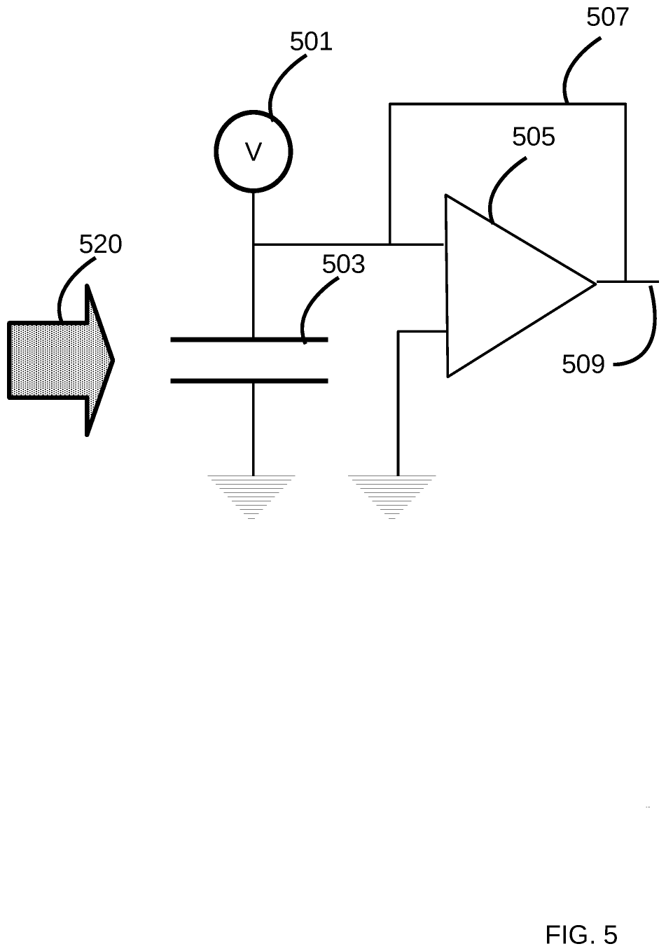

FIG. 5 is an example of an US transceiver configured to detect an ultrasound back scatter signal. The US transceiver (503) is depicted as a capacitor with a capacitance dependent on the local air pressure generated by an ultrasound backscatter signal (507) on the US transceiver (503). The capacitor is biased with a voltage source V (501) and connected to an amplifier (505). When the capacitance of the capacitor changes, the amplifier is configured to generate a current in the output electrical connection (510). The output signal is a current proportional to the ultrasound backscatter signal (507) impinging on the US transceiver (503). Examples of bias voltages of the voltage source V (501) include but are not limited to 5V; 10V; 1-10V; 11-20V; 21-30V; less than 50V. In one example, the output signal is processed either as an analog signal using analog filters, mixers or other electronic components or as a digital filter where the signal is filtered, sampled and further processed using digital signal processing techniques. The physical or biometric parameter estimation are derived from the processed output signal. In one example the transmitted signal is a time limited broad band pulse or pulse train and the received signal is a time delayed pulse or group of pulses. The time delay is estimated from any of but not limited to the pulse leading edge; pulse correlation function; threshold crossing function or other time estimation method. The time delay is indicative of any of but not limited to distance form tympanic membrane, volume of ear canal, hand, ear, finger, or other body item gesture recognition. Additional parameters relating to a change in the time delay relate to body temperature through changes in the air speed.

In an alternative example the transmitted signal is a narrow band signal. Examples of narrow band signals include but are not limited to time limited pulses with least 10 or more cycles of the carrier ultrasound frequency; time varying frequency signals, other continuous or semi continuous ultrasound signals or repetition of such signals. The estimation of the time delay is obtained by any of but not limited to, phase delay estimation, frequency correlation (typically in FMCW configurations), time and frequency estimation. In a further example additional physical or biometric parameter are estimated by changes in the return signal frequency due to a doppler shift. Examples of such parameters include but are not limited to heart rate; heart rate variability; ear effusivity; infections; liquids in ear or surrounding tissue.

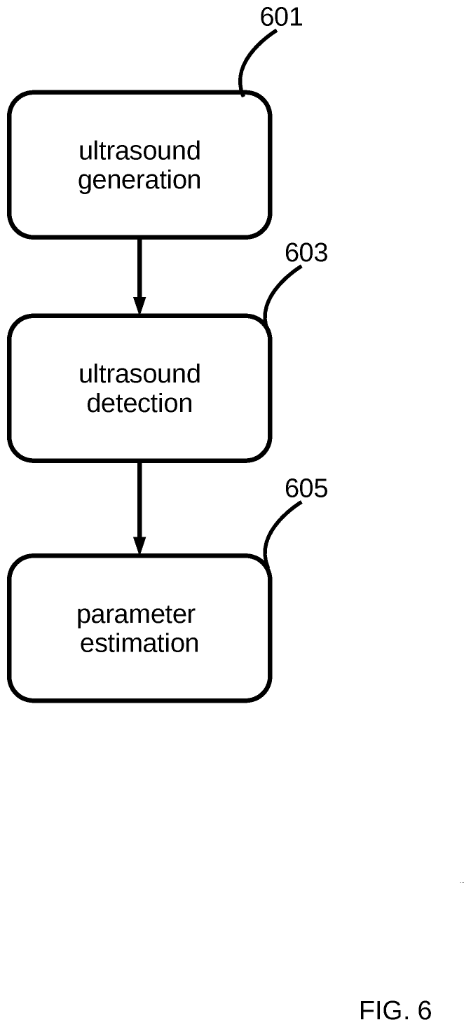

FIG. 6 is an example of a method for measuring a physical parameter using ultrasound back reflection. An ultrasound signal is generated (601) by an ultrasound transmitter. In one example an ultrasound modulated speaker generates residual ultrasound in the ear which is used for backscattering measurement. An ultrasound receiver for example as described in FIG. 3 and FIG. 4 detects the ultrasound backscatter signal. In further example a method for parameter extraction (605) from the detected ultrasound backscatter is implemented as an algorithm in a processing unit. Examples of processing unit include but are not limited to controller, processor, digital processor, neural network or similar processing units.

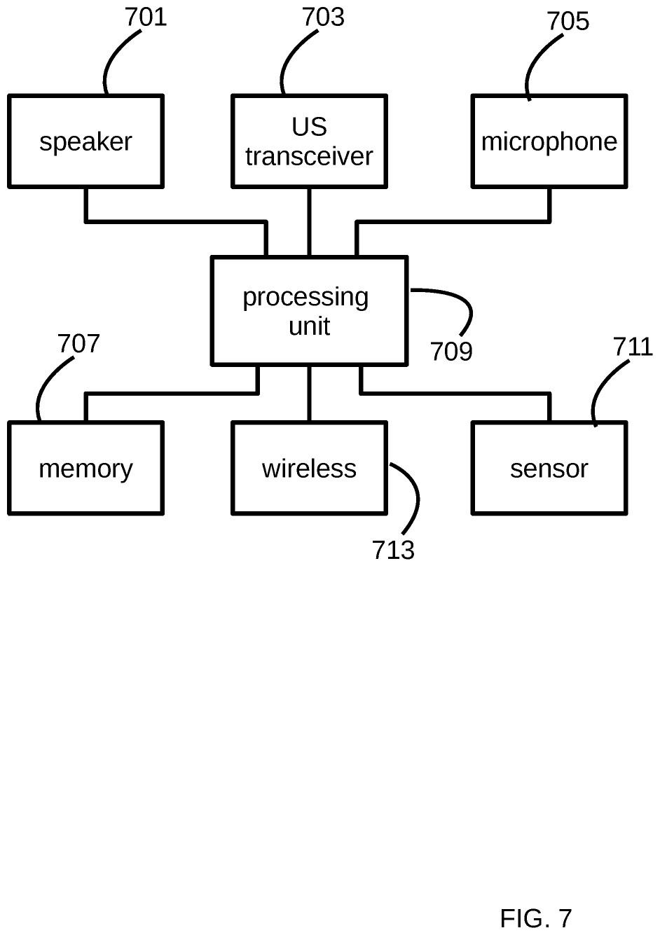

FIG. 7 is an example of an earphone system. An earphone system may include any of but is not limited to; a processing unit (709) speaker (701); US transceiver (703); microphone (705); memory unit (707); wireless or wired communication unit (713); additional sensors (711). Examples of processing unit (709) include any of but are not limited to; controller; DSP; MCU; GPU; Machine learning module; Al module or custom ASIC or FPGA or combinations of these. Examples of speakers (701) include any of but are not limited to; modulated ultrasound speakers; balanced armature; dynamic voice coil; MEMS membrane speakers; pump speakers; electrostatic speakers; ionic speakers; piezo speakers. Examples of US transceivers (703) include any of but are not limited to MEMS CMUT; MEMS PMUT; modulated ultrasound speaker with or without dedicated receiver membranes. Examples of microphones (705) include any of electret microphone and MEMS capacitive or piezo microphone. Microphones may be either digital or analogue. Examples of memory unit (707) include any of but are not limited to RAM, NRAM, ROM, NOR, volatile or nonvolatile memory. Examples of wireless or wired communication unit (713) include any of but not limited to Bluetooth; Zigbee; Wi-Fi; cable connection; proprietary wireless protocol. Examples of sensors (711) include any of but are not limited to; accelerometer; gyro; pressure sensor; IR transmitter; IR receiver; proximity sensor; RF sensor; temp sensor; gyro; or combinations of said sensors.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to disclosures containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An audio system comprising:
a modulated ultrasound audio driver comprising at least one ultrasound emitter configured to emit an ultrasound signal; at least one acoustic mixer configured to generate an audio signal from the ultrasound signal; and at least one ultrasound receiver configured to detect an ultrasound signal and generate a corresponding electrical signal; and
a processing unit configured to process the electrical signal and estimate one or more physical or biometric parameters from the electrical signal.

2. The audio system according to claim 1, wherein the ultrasound receiver is comprised of at least one dedicated membrane in the modulated ultrasound audio driver.

3. The audio system according to claim 1, wherein the physical or biometric parameter is any of but not limited to distance of a speaker from tympanic membrane; a volume of an ear canal, a length of ear canal; a heart beat from reflection of veins in ear, or jugular vein, or from vibrations induced by blood flow and manifested in tympanic membrane; effusivity of ear; viscosity of ear fluid; infections or other problems in an inner ear; vibrations in tympanic membrane resulting from external noise or internal noise; blood flow; temperature in an ear canal and relation to body temperature; proximity to an ear, ear canal or tympanic membrane.

4. A method for physical or biometric parameter estimation comprising:
generating an ultrasound signal from a modulated ultrasound speaker;
detecting a backscattered ultrasound signal with a dedicated receiver and generating an electric signal; and
processing the electric signal and generating an estimation of a physical or biometric parameter.

5. The method for physical or biometric parameter estimation according to claim 4, wherein the physical or biometric parameters is any of but not limited to a distance of speaker from tympanic membrane; a volume of an ear canal, a length of an ear canal; a heart beat from reflection of veins in an ear, or jugular vein, or from vibrations induced by blood flow and manifested in a tympanic membrane; effusivity of an ear; viscosity of ear fluid; infections or other problems in an inner ear; vibrations in tympanic membrane resulting from external noise or internal noise; blood flow; temperature in an ear canal and relation to body temperature; proximity to an ear, ear canal or tympanic membrane.
